(19) United States
(12) Patent Application Publication
(10) Pub. No.: US 2024/0398691 A1
(43) Pub. Date: Dec. 5, 2024

(54) Rinse-Free Nutrient Rich Rinse

(71) Applicant: AIH PERSONAL CARE PRO, LLC, Schaumburg, IL (US)

(72) Inventors: Abdessettar Ibourki, Schaumburg, IL (US); Sharon Martiny, Alton (GB); Khadija Ibourki, Khouribga (MA)

(73) Assignee: AIH PERSONAL CARE PRO, LLC, Schaumburg, IL (US)

(21) Appl. No.: 18/620,027

(22) Filed: Mar. 28, 2024

Related U.S. Application Data
(60) Provisional application No. 63/469,867, filed on May 31, 2023.

(51) Int. Cl. A61K 8/9789; A61K 8/34; A61K 8/362; A61Q 5/00

(52) U.S. Cl. CPC A61K 8/9789; A61K 8/345; A61K 8/362; A61Q 5/00

(57) ABSTRACT

A rinse-free nutrient rich rinse is provided that includes an herbal extract blend of natural herbal active ingredients. The nutrient-rich rinse can be applied to dry or damp hair to provide enhanced moisturization and glow and need not be rinsed or washed away. The components work together to moisturize dry, brittle hair and contribute to a shiny, luscious appearance. The rinse-free nutrient rich rinse can be used alone or in combination with an all-natural conditioner hair treatment and an herbal cleansing shampoo.

### RELATED APPLICATIONS

This patent application claims priority to U.S. Provisional Application 63/469,867, filed on May 31, 2023, the disclosure of which is incorporated herein by reference.

### FIELD OF THE INVENTION

This invention is directed to an herbal rinse that can be applied post-shampoo or at any time to dry or damp hair. Because the herbal rinse is formed using natural herbal active ingredients that are healthy to the hair and scalp, it does not have to be washed or rinsed away after application. The nutrient rich herbal rinse can be applied alone or in sequence with an all-natural conditioner hair treatment and an herbal cleansing shampoo to help eliminate dry, brittle hair and replace it with healthy hair and luscious locks.

### BACKGROUND OF THE INVENTION

Hair treatment products have been developed over the years with the objective of providing cleaner, shinier, and healthier looking hair. Many of these products contain synthetic ingredients that are effective in shampooing, cleaning, and rinsing the hair, but which can result in dry scalp, dandruff and dry or brittle hair Additionally, the widespread use of synthetic ingredients can result in contamination of rivers, streams, and groundwater, causing harmful effects to fish and other wildlife. There is a need or desire for hair treatment formulas (including rinses) that are not only effective in producing cleaner and shinier hair, but also avoid damage to the users' hair and scalp, and which do not cause harm to the surrounding environment.

### SUMMARY OF THE INVENTION

This invention is directed to a rinse-free nutrient rich rinse that can be applied alone or in sequence with other all-natural pre-shampoo hair conditioner and herbal shampoo. The term “rinse-free” means that the nutrient rich rinse does not have to be washed or rinsed away after application. The rinse-free nutrient rich rinse contains all natural active ingredients that promote healthy, shiny hair with luscious locks and have environmental advantages.

The rinse-free nutrient rich rinse can include some or all of the following ingredients, which can be present in the amounts described herein.

Water base: preferably deionized water.

Herbal Extracts: ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, thymus vulgaris extract.

Natural Oil: glycerin.

Performance and processing aids: citric acid, centrimonium chloride, sodium benzoate, potassium sorbate.

In one embodiment, it is a feature and advantage of the invention to provide nutrient rich rinse for hair, including an herbal extract blend;
wherein the herbal extract blend includes three or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract.

In another embodiment, it is a feature and advantage of the invention to provide a nutrient rich rinse for hair, that includes:
about 70% to about 96% by weight water;
about 2% to about 20% by weight of an herbal extract blend component;
about 1% to about 8% by weight of a natural oil component; and
about 1% to about 10% by weight of a performance and processing aid component.

In another embodiment, it is a feature and advantage of the invention to provide a nutrient rich rinse for hair, that includes:
about 84% to about 90% by weight water;
about 4% to about 10% by weight of an herbal extract component including two or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract, and combinations thereof;
about 2% to about 7% by weight of a natural oil component including glycerin; and
about 2% to about 8% by weight of a performance and processing aid component including two or more of citric acid, centrimonium chloride, sodium benzoate, and potassium sorbate.

Additional embodiments of the invention are provided in the following Detailed Description.

### DETAILED DESCRIPTION OF THE INVENTION

The rinse-free nutrient rich rinse is especially designed to provide the user with shiner, healthier hair with a luscious appearance, and is environmentally friendly. The rinse-free nutrient rich rinse can be used alone as a rinse, or as part of an overall hair treatment sequence. One example of an overall hair treatment sequence can include first treating the hair with a pre-shampoo argan oil blend hair treatment as described in co-pending U.S. patent application Ser. No. ______, entitled “Argan Oil Blend Hair Treatment,” subsequently shampooing the hair with a herbal cleansing shampoo as described in co-pending U.S. patent application Ser. No. ______, entitled “Herbal Cleansing Shampoo,” and separately treating the clean hair with the rinse-free nutrient rich rinse. Both co-pending applications are filed on even date herewith and are incorporated herein by reference.

In addition to water, the rinse-free nutrient rich rinse can include an herbal extract blend component, a surfactant component, a natural oil component, and a performance and processing aid component. The water component is preferably deionized water and can constitute about 70% to about 96% by weight of the rinse-free nutrient rich rinse, or about 77% to about 93% by weight, or about 84% to about 90% by weight. The herbal extract blend component can constitute about 2% to about 20% by weight of the rinse-free nutrient rich rinse, or about 3% to about 15% by weight, or about 4% to about 10% by weight. The surfactant component can constitute 0% to about 5% by weight of the herbal cleansing shampoo, or about 0.1% to about 3% by weight, or about 0.2% to about 1% by weight. The natural oil component can constitute about 1% to about 8% by weight of the rinse-free nutrient rich rinse, or about 2% to about 7% by weight, or about 3% to about 6% by weight. The performance and processing aid component can constitute 1% to about 10% by weight of the rinse-free nutrient rich rinse, or about 1.5% to about 9% by weight, or about 2% to about 8% by weight. As used herein, the phrase “performance and processing aid” includes ingredients that stabilize and maintain the performance of other ingredients in the herbal cleansing shampoo, ingredients that enhance the performance of other ingredients, and ingredients that aid in the manufacture, processing, and use of the rinse-free nutrient rich rinse.

The herbal extract blend component of the rinse-free nutrient rich rinse can include one or more, two or more, three or more, four or more, five or more, six or more, or seven or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract. Each of these herbal extract ingredients can, when present, constitute about 0.2% to about 10% by weight of the rinse-free nutrient rich rinse, or about 0.4% to about 5% by weight, or about 0.6% to about 2% by weight.

The natural oil component of the rinse-free nutrient rich rinse is suitably a water-soluble oil. Suitable water-soluble oils include without limitation glycerin, alone or in combination with other natural oils. Glycerin not only moisturizes the hair and scalp, but also promotes thicker hair and new hair growth and alleviates dandruff-related symptoms.

The performance and processing aid component can include one or more, or two or more, or all three of citric acid, aqueous cenrtimonium chloride, sodium benzoate, and potassium sorbate. Each of these ingredients can, when present, constitute about 0.1% to about 8% by weight of the rinse-free nutrient rich rinse, or about 0.2% to about 5% by weight, or about 0.3% to about 3% by weight. While sodium benzoate and potassium sorbate mainly act as preservatives by hindering microbial and fungal growth, citric acid enhances the performance of other ingredients in the rinse-free nutrient rich rinse. Centrimonium chloride currently has a regulatory upper limit of 0.3% for leave-on products, allowing a 30% aqueous blend to be used at up to 1% by weight of the rinse-free nutrient rich rinse.

The following Table 1 shows an example of an herbal cleansing shampoo formulation useful in the invention.

TABLE 1
Example Rinse-Free Nutrient Rich Rinse Formula (% By Weight)

| | Broad | Intermediate | Narrow |
|---|---|---|---|
| Component | Range | Range | Range |
| Deionized Water | 70-96 | 77-93 | 84-90 |
| Herbal Extract Blend Component | 2-20 | 3-15 | 4-10 |
| ocmium basilicum leaf extract | 0.2-10 | 0.4-5 | 0.6-2 |
| citrus aurantium amara flower extract | 0.2-10 | 0.4-5 | 0.6-2 |
| rosmarius officinalis leaf extract | 0.2-10 | 0.4-5 | 0.6-2 |
| chamomilia recutia flower extract | 0.2-10 | 0.4-5 | 0.6-2 |
| lavandula angustifolia flower extract | 0.2-10 | 0.4-5 | 0.6-2 |
| artemisia vulgaris herb extract | 0.2-10 | 0.4-5 | 0.6-2 |
| ocmium basilicum seed extract | 0.2-10 | 0.4-5 | 0.6-2 |
| eugenia caryophyllus bud extract | 0.2-10 | 0.4-5 | 0.6-2 |
| thymus vulgaris extract | 0.2-10 | 0.4-5 | 0.6-2 |
| Natural Oil Component | 1-8 | 2-7 | 3-6 |
| glycerin | 1-8 | 2-7 | 3-6 |
| Performance and Processing Aid | 1-10 | 1.5-9 | 2-8 |
| Component | | | |
| citric acid | 0.1-8 | 0.2-5 | 0.3-3 |
| aqueous cetrimonium chloride (30%) | 0.1-8 | 0.2-5 | 0.3-1* |
| sodium benzoate | 0.1-8 | 0.2-5 | 0.3-3 |
| potassium sorbate | 0.1-8 | 0.2-5 | 0.3- |
| TOTAL | 100 | 100 | 100 |

*Centrimoium chloride currently has a regulatory upper limit of 0.3% excluding water.

No special order of addition or mixing is required for blending the ingredients of the rinse-free nutrient rich rinse. The ingredients can be added and mixed all at once to any suitable agitated mixture, or they can be premixed as separate sub-components that can then be blended together. The ingredients can be mixed thoroughly at ambient temperature and pressure and can be filled into bottles or other suitable containers for use by consumers. In one embodiment, the preservatives can first be mixed with the deionized water. The remaining ingredients can then be added in the order listed above, while mixing each one thoroughly until a uniform mixture is obtained.

Several ingredients of the herbal cleansing shampoo have advantageous properties that, together and alone, contribute to shiny, healthy, thicker, and younger-looking hair and a healthy scalp. Referring to the ingredients of the herbal extract blend component, ocmium basilicum leaf extract contains antioxidants that help to nourish and strengthen hair follicles, promoting healthy hair growth. It also has antibacterial properties that help to prevent scalp infections and dandruff, leaving the hair clean and healthy. Citrus aurantium amara flower extract moisturizes the hair shaft and prevents the hair from becoming brittle, dull, or scratchy. Rosmarius officinalis leaf extract is believed to help heal nerve endings and may rejuvenate nerves in the scalp, aiding hair growth. Chamomilia recutia flower extract helps promote hair strength, helps prevent split ends, and lightens hair strands. Lavandula angustifolia flower extract can strengthen hair follicles, promote hair growth, and reduce frizz. Artemisia vulgaris herb extract can strengthen hair roots, reduce hair loss, and improve scalp health. Ocmium basilicum seed extract moisturizes the hair and scalp and can reduce hair loss. Eugenia caryophyllus bud extract helps to combat dry hair and split ends and promotes hair follicle activity to encourage hair growth. Thymus vulgaris extract eases inflammation affecting the scalp and hair follicles to help combat dandruff.

Citric acid helps remove buildup of debris in hair, resulting in shinier hair, and enhances the performance of the remaining ingredients. Glycerin combats dandruff and split ends by moisturizing the scalp and strengthening and moisturizing the hair. Centrimonium chloride has antimicrobial and anti-static properties for hair. Sodium benzoate and potassium sorbate act as preservatives in the rinse-free nutrient rich rinse and prevent the growth of microbes and fungi.

The rinse-free nutrient rich rinse can be applied to damp or dry hair once per week or as needed, using eight or more sprays for shoulder length hair with average thickness. Once applied, the rinse-free nutrient rich rinse can be massaged gently into the scalp and hair. The hair can optionally be dried using a hair dryer but does not need to be rinsed after application of the rinse-free nutrient rich rinse. Prior to using the rinse, the user's hair can optionally be treated with a conditioner, for example using an argan oil blend hair treatment as described in the above-identified co-pending U.S. patent application Ser. No. ______, entitled “Argan Oil blend Hair Treatment” and, can be shampooed using an herbal cleansing shampoo as described in co-pending U.S. patent application Ser. No. ______, entitled “Herbal Cleansing Shampoo.”

The embodiments of the invention described herein are exemplary. Various modifications and improvements can be made without departing from the spirit and scope of the invention. The scope of the invention is defined in the appended claims, and all changes that fall within the meaning and range of equivalency are intended to be embraced therein.

1. A nutrient rich rinse for hair, comprising an herbal extract blend;
wherein the herbal extract blend includes three or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract.

2. The nutrient rich rinse of claim 1, wherein the herbal extract blend comprises five or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract.

3. The nutrient rich rinse of claim 1, wherein the herbal extract blend comprises seven or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract.

4. The nutrient rich rinse of claim 1, comprising:
about 2% to about 20% by weight of the herbal extract blend;
about 70% to about 96% by weight water;
about 1% to about 8% by weight of a natural oil component; and
about 1% to about 10% by weight of a performance and processing aid component.

5. The nutrient rich rinse of claim 4, wherein the natural oil component comprises glycerin and the performance and processing aid component comprises citric acid.

6. The nutrient rich rinse of claim 5, wherein the performance and processing aid component further comprises at least one of potassium sorbate and sodium benzoate.

7. The nutrient rich rinse of claim 4, comprising:
about 3% to about 15% by weight of the herbal extract blend;
about 77% to about 93% by weight of the water;
about 2% to about 7% by weight of the natural oil component; and
about 1.5% to about 9% by weight of the performance and processing aid component.

8. The nutrient rich rinse of claim 4, comprising:
about 4% to about 10% by weight of the herbal extract blend;
about 84% to about 90% by weight water;
about 3% to about 6% by weight of the natural oil component; and
about 2% to about 8% by weight of the performance and processing aid component.

9. The nutrient rich rinse of claim 4, wherein the natural oil component comprises glycerin.

10. The nutrient rich rinse of claim 9, wherein the performance and processing aid component is selected from the group consisting of citric acid, centrimonium chloride, sodium benzoate, potassium sorbate, and combinations thereof.

11. A nutrient rich rinse for hair, comprising:
about 70% to about 96% by weight water;
about 2% to about 20% by weight of an herbal extract component;
about 1% to about 8% by weight of a natural oil component; and
about 1% to about 10% by weight of a performance and processing aid component.

12. The nutrient rich rinse of claim11, wherein the water comprises deionized water.

13. The nutrient rich rinse of claim 11, wherein the herbal extract component is selected from the group consisting of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract, and combinations thereof.

14. The nutrient rich rinse of claim 11, wherein the herbal extract component is present in an amount of about 3% to about 15% by weight of the nutrient rich rinse and includes three or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract, and combinations thereof.

15. The nutrient rich rinse of claim 11, wherein the herbal extract component is present in an amount of about 4% to about 10% by weight of the nutrient rich rinse and includes five or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract, and combinations thereof.

16. The nutrient rich rinse of claim 11, wherein the natural oil component comprises glycerin.

17. The nutrient rich rinse of claim 11, wherein the performance and processing aid component is selected from the group consisting of citric acid, centrimonium chloride, sodium benzoate, potassium sorbate, and combinations thereof.

18. A nutrient rich rinse for hair, comprising:
about 84% to about 90% by weight water;
about 4% to about 10% by weight of an herbal extract component including two or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract, and combinations thereof;
about 2% to about 7% by weight of a natural oil component including glycerin; and
about 2% to about 8% by weight of a performance and processing aid component including two or more of citric acid, centrimonium chloride, sodium benzoate, and potassium sorbate.

19. The nutrient rich rinse of claim 18, wherein the herbal extract component includes four or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract, and combinations thereof.

20. The nutrient rich rinse of claim 18, wherein the herbal extract component includes six or more of ocmium basilicum leaf extract, citrus aurantium amara flower extract, rosmarius officinalis leaf extract, chamomilia recutia flower extract, lavandula angustifolia flower extract, artemisia vulgaris herb extract, ocmium basilicum seed extract, eugenia caryophyllus bud extract, and thymus vulgaris extract, and combinations thereof.
